(19) United States
(12) Patent Application Publication
(10) Pub. No.: US 2015/0359992 A1
(43) Pub. Date: Dec. 17, 2015

(54) ELECTRONIC DEVICE, METHOD, AND COMPUTER PROGRAM PRODUCT

(71) Applicant: Kabushiki Kaisha Toshiba, Tokyo (JP)

(72) Inventors: Takashi SUDO, Fuchu Tokyo (JP); Yasuhiro KANISHIMA, Suginami Tokyo (JP); Takaya MATSUNO, Kunitachi Tokyo (JP)

(21) Appl. No.: 14/659,423

(22) Filed: Mar. 16, 2015

Related U.S. Application Data
(60) Provisional application No. 62/013,151, filed on Jun. 17, 2014.

(51) Int. Cl. A61M 21/00; A61B 5/00

(52) U.S. Cl. CPC A61M 21/00; A61B 5/4812; A61B 5/4809; A61B 2562/0219; A61M 2021/0022; A61M 2021/0027; A61M 2021/0083; A61M 2205/3303; A61M 2230/63

(57) ABSTRACT

According to an embodiment, an electronic device includes, for example, an indicator, a sensor, and circuitry. The indicator is configured to notifying a user by using at least one of a sound and vibration. The sensor is configured to detect first information regarding a body of a user, the first information possibly subject to disturbance during notifying by the indicator. The circuitry is configured to replace the first information during notifying by the indicator with second information different from the first information.

### CROSS-REFERENCE TO RELATED APPLICATIONS

This application claims the benefit of U.S. Provisional Patent Application No. 62/013,151, filed Jun. 17, 2014, the entire contents of which are incorporated herein by reference.

### FIELD

The embodiment disclosed herein generally relates to an electronic device, a method, and a computer program product.

### BACKGROUND

Electronic devices such as a wearable device in a form of a wristband include a terminal that detects, by use of an accelerometer, body movement amount of a user who wears the wearable device, and performs sleep analysis by use of the detected amount of body movement to acquire first information relating to the body, for example, in which sleep stages, wakefulness, light sleep, or deep sleep, the body is. When determining that the body is in a light sleep stage, the wearable device can execute a first operation, such as an alarm function that causes rumbling to wake the user up, that may cause disturbance to the first information.

However, the rumbling of the alarm function affects the detection result of the accelerometer in some cases, thereby lowering the accuracy of the sleep analysis performed by the wearable device.

### BRIEF DESCRIPTION OF THE DRAWINGS

A general architecture that implements the various features of the invention will now be described with reference to the drawings. The drawings and the associated descriptions are provided to illustrate an embodiment of the invention and not to limit the scope of the invention.

FIG. 1 is a diagram illustrating an example of a configuration of a body information acquisition system according to an embodiment;

FIG. 2 is a block diagram illustrating an example of a functional configuration of a wearable device according to the present embodiment;

FIG. 3 is a flowchart illustrating an example of the procedure for acquiring body information by the wearable device according to the present embodiment;

FIG. 4 is a diagram illustrating an example of time variation of body movement amount calculated by the wearable device according to the present embodiment;

FIG. 5 is a diagram illustrating an example of acquisition processing for acquiring the body information by the wearable device according to the present embodiment;

FIG. 6 is a diagram illustrating a display example of analysis results displayed on a host device according to the present embodiment;

FIG. 7 is a diagram illustrating another display example of the analysis results displayed on the host device according to the present embodiment;

FIG. 8 is a diagram illustrating an example of replacement of other types of body information with certain second information by the wearable device according to the present embodiment;

FIG. 9 is a diagram illustrating an example of acquisition processing for acquiring the body information by the wearable device according to the present embodiment;

FIG. 10 is a diagram illustrating another example of the acquisition processing for acquiring the body information by the wearable device according to the present embodiment;

FIG. 11 is a diagram illustrating an example of the relation between the intensity of a signal of acceleration data and the frequency of the signal of the acceleration data that varies over time when the wearable device according to the present embodiment acquires sleep stages of a user; and

FIG. 12 is a diagram illustrating an example of the relation between the intensity of a signal of acceleration data and the frequency of the signal of the acceleration data when the wearable device according to the present embodiment acquires the number of steps of the user.

### DETAILED DESCRIPTION

In general, according to an embodiment, an electronic device includes an indicator, a sensor, and circuitry. The indicator is configured to notify a user by using at least one of a sound and vibration. The sensor is configured to detect first information regarding a body of a user, the first information possibly subject to disturbance during notifying by the indicator. The circuitry is configured to replace the first information during notifying by the indicator with second information different from the first information.

The following describes a body information acquisition system employing the electronic device, a method, and a computer program product according to the present embodiment with reference to the accompanying drawings.

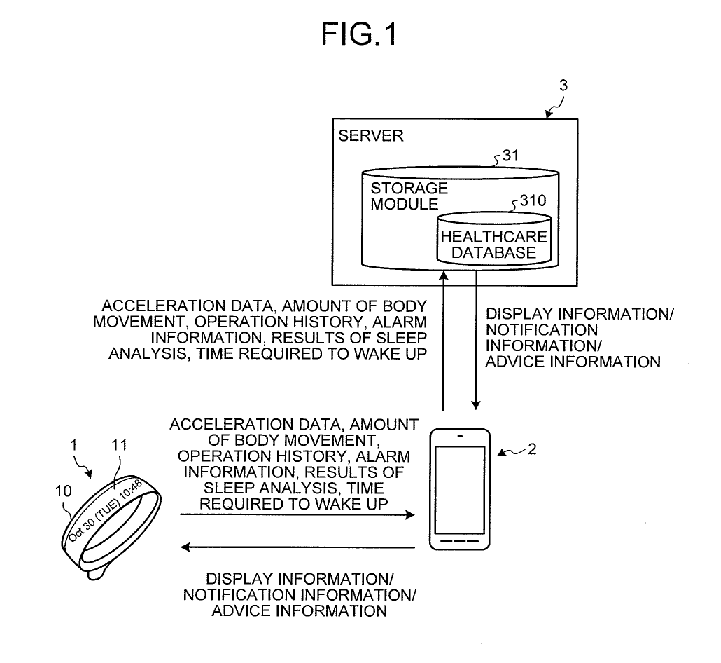

FIG. 1 is a diagram illustrating an example of a configuration of the body information acquisition system according to the present embodiment. As illustrated in FIG. 1, the body information acquisition system according to the present embodiment includes a wearable device 1, a host device 2, and a server 3.

The wearable device 1 (an example of the electronic device) includes a housing 10 that can be worn by a user on a part of the user's body. The wearable device 1 includes a display screen 11 configured with, for example, a liquid crystal display (LCD) or an organic electro luminescence (EL) display, and configured to display various kinds of information such as time. The wearable device 1 transmits, to the host device 2, various kinds of information such as body information of the user, operation history of the user, alarm information, and time required for the user to wake up.

The body information is information relating to the body of a user who wears the wearable device 1, and is an example of first information that may be subject to disturbance while alarm to be described later is being executed. The body information includes, for example, data on acceleration (hereinafter referred to as acceleration data) detected by an accelerometer 12 provided in the housing 10, an amount of body movement of the user acquired on the basis of the acceleration data, results of sleep analysis based on the amount of body movement, and results of analysis on activity information (for example, the number of steps the user has taken, the user is walking, at rest, moving by bicycle, or moving by train) based on the amount of body movement. The results of the sleep analysis include sleep time at which the user fell asleep, wake time at which the user woke up, an amount of sleep time from the time at which the user fell asleep to the time at which the user woke up, light-sleep time that is the sum of amounts of time of light sleep, and deep-sleep time that is the sum of amounts of time of deep sleep.

The alarm information is information relating to alarm that is an operation for notifying the user and is an example of the first operation causing disturbance to the body information, and the alarm information is such as alarm set time (an example of a certain first timing) at which alarm (in the present embodiment, vibration and sound, that is, rumbling) is executed, a target number of steps (another example of the certain first timing) set in advance at which the alarm is executed, and setting or cancelation of the alarm. The time required for the user to wake up is information relating to a time period from the time at which the alarm is executed to the time at which the wearable device 1 acquires wakefulness as the sleep stage on the basis of the body information.

The wearable device 1 receives, from the host device 2, various kinds of information such as display information displayed on the display screen 11, notification information (for example, the arrival of a mail, a phone call, and an emergency call at the host device 2) to the user who wears the wearable device 1, advice information relating to advice for the user obtained from the analysis of the body information.

The host device 2 is configured with a smartphone or a tablet device, and receives various kinds of information such as the body information, the operation history, the alarm information, and the time required for the user to wake up from the wearable device 1. The host device 2 transmits various kinds of information such as the body information, the operation history, the alarm information, and the time required for the user to wake up received from the wearable device 1 to the server 3. The host device 2 receives various kinds of information such as the display information, the notification information, and the advice information from the server 3. The host device 2 transmits various kinds of information such as the display information, the notification information, and the advice information received from the server 3 to the wearable device 1.

The server 3 includes a storage module 31 that can store various kinds of information received from the host device 2. The storage module 31 includes a healthcare database 310 that can store such as the body information, the operation history, the alarm information, and the time required for the user to wake up received from the host device 2.

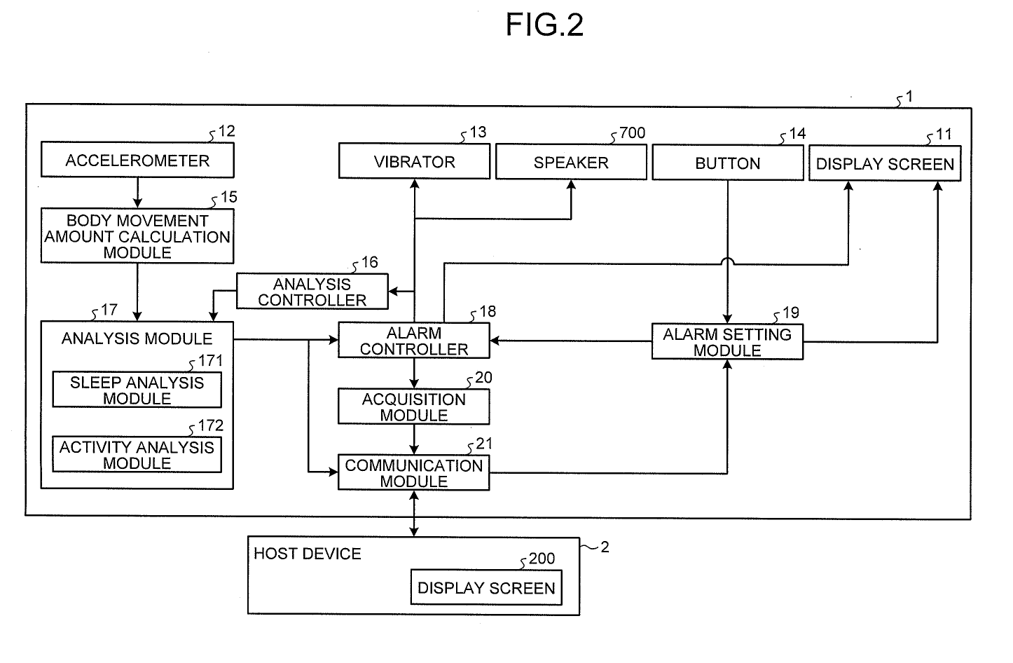

Described next is a functional configuration of the wearable device 1 according to the present embodiment with reference to FIG. 2. FIG. 2 is a block diagram illustrating an example of the functional configuration of the wearable device according to the present embodiment.

As illustrated in FIG. 2, the wearable device 1 according to the present embodiment includes the accelerometer 12, a vibrator 13, a button 14, the display screen 11, a body movement amount calculation module 15, an analysis controller 16, an analysis module 17, an alarm controller 18, an alarm setting module 19, an acquisition module 20, a communication module 21, and a speaker 700.

The accelerometer 12 is provided in the housing 10 and is configured to detect (acquire), as an example of the body information, acceleration data of the housing 10 worn by the user. The accelerometer 12 is an example of the sensor that may be subject to disturbance affecting the acceleration data while the alarm is being executed. The body movement amount calculation module 15 is configured to calculate (acquire) an amount of body movement of the user as an example of the body information on the basis of the acceleration data detected by the accelerometer 12. The body movement amount calculation module 15 is an example of the sensor that may be subject to disturbance affecting the amount of body movement while the alarm is being executed.

The analysis module 17 acquires the body information on the basis of the amount of body movement calculated by the body movement amount calculation module 15 or the acceleration data detected by the accelerometer 12. In the present embodiment, the analysis module 17 includes a sleep analysis module 171 and an activity analysis module 172. The sleep analysis module 171 is configured to acquire information relating to sleep stages of the user as an example of the body information on the basis of the amount of body movement calculated by the body movement amount calculation module 15. The sleep analysis module 171 is an example of the sensor that may be subject to disturbance affecting the sleep stages while the alarm is being executed. In the present embodiment, the sleep analysis module 171 acquires a sleep stage of the user (wakefulness, light sleep, or deep sleep) on the basis of an amount of body movement calculated by the body movement amount calculation module 15. The activity analysis module 172 is configured to estimate (acquire) activity information of the user (for example, the number of steps the user has taken, the user is walking, at rest, moving by bicycle, or moving by train) as an example of the body information on the basis of the acceleration data detected by the accelerometer 12. The activity analysis module 172 is an example of the sensor that may be subject to disturbance affecting the activity information while the alarm is being executed.

The vibrator 13 is an example of the indicator configured to execute the alarm (vibration) as the first operation for notifying the user. In the present embodiment, the vibrator 13 can execute the alarm causing disturbance that may affect the body information (in the present embodiment, acceleration data, an amount of body movement, the sleep stages, and the activity information) at the first timing such as the alarm set time set by the alarm setting module 19 to be described later, and a timing at which the number of steps acquired by the activity analysis module 172 reaches the target number of steps. The vibrator 13 can also execute the alarm (vibration) when the wearable device 1 receives the notification information from the host device 2. The speaker 700 is an example of the indicator configured to execute the alarm (output of sound) as the first operation for notifying the user. The speaker 700 can execute the alarm (output of sound) causing disturbance that may affect the body information (in the present embodiment, acceleration data, an amount of body movement, the sleep stages, and the activity information) at the first timing such as the alarm set time set by the alarm setting module 19 to be described later, and a timing at which the number of steps acquired by the activity analysis module 172 reaches the target number of steps. The speaker 700 can also execute the alarm (output of sound) when the wearable device 1 receives the notification information from the host device 2. The button 14 can execute vibration on a user's operation as an example of the first operation.

The analysis controller 16 controls acquisition of body information by the analysis module 17. In the present embodiment, the analysis controller 16 is an example of the processor that replaces the sleep stage acquired by the sleep analysis module 171 when the vibrator 13 and the speaker 700 is executing the alarm with a certain sleep stage (in the present embodiment, light sleep) that is an example of the second information different from the acquired sleep stage. This configuration can prevent disturbance caused by the vibrator 13 and the speaker 700 from being included in the sleep stages acquired by the sleep analysis module 171, thereby improving accuracy of the sleep stages acquired by the sleep analysis module 171. This configuration can also prevent disturbance from being included in the sleep stages acquired by the sleep analysis module 171 even when the vibrator 13 and the speaker 700 are disposed near the accelerometer 12 in the housing 10. Accordingly, the accelerometer 12, the vibrator 13, and the speaker 700 can be disposed more freely. The configuration described above does not require processing for eliminating disturbance from the sleep stages acquired by the sleep analysis module 171, the acceleration data detected by the accelerometer 12, or the amount of body movement calculated by the body movement amount calculation module 15, so that energy consumption of the wearable device 1 is reduced and the battery can supply power for a longer time.

The alarm setting module 19 sets the alarm set time and the target number of steps in accordance with a user's operation on the button 14 or the alarm information received from the host device 2 via the communication module 21 to be described later. The alarm controller 18 causes the vibrator 13 and the speaker 700 to execute the alarm at the first timing such as the alarm set time or a timing at which the number of steps acquired by the activity analysis module 172 reaches the target number of steps. In the present embodiment, when the sleep analysis module 171 is acquiring the sleep stages of the user, the first timing is the earlier timing of the two timings, the alarm set time set by the alarm setting module 19 and a timing at which the sleep analysis module 171 acquires light sleep as the sleep stage of the user for the first time in a certain time period prior to the alarm set time. Although, in the present embodiment, the alarm controller 18 causes the vibrator 13 to execute vibration and the speaker 700 to execute output of sound as the alarm, the embodiment is not limited to this. For example, the alarm controller 18 may cause the vibrator 13 to execute vibration, or the speaker 700 to execute output of sound as the alarm.

The acquisition module 20 is an example of the processor that acquires, as time information required for the user to wake up, information relating to a time period from the execution of alarm by the vibrator 13 to a timing at which the sleep analysis module 171 acquires wakefulness as the sleep stage of the user. The acquisition module 20 outputs the acquired time information to the host device 2 via the communication module 21.

The communication module 21 transmits various kinds of information such as the body information, the operation history, the alarm information, and the time required for the user to wake up to the host device 2. The communication module 21 receives various kinds of information such as the display information, the notification information, the advice information, and the alarm information from the host device 2.

The host device 2 includes a display screen 200 that can display various kinds of information such as the body information, the operation history, the alarm information, and the time required for the user to wake up that are received from the wearable device 1. With this configuration, the host device 2 can show the user how much time was required to wake up, thereby helping the user recognize the user's current lifestyle to improve it.

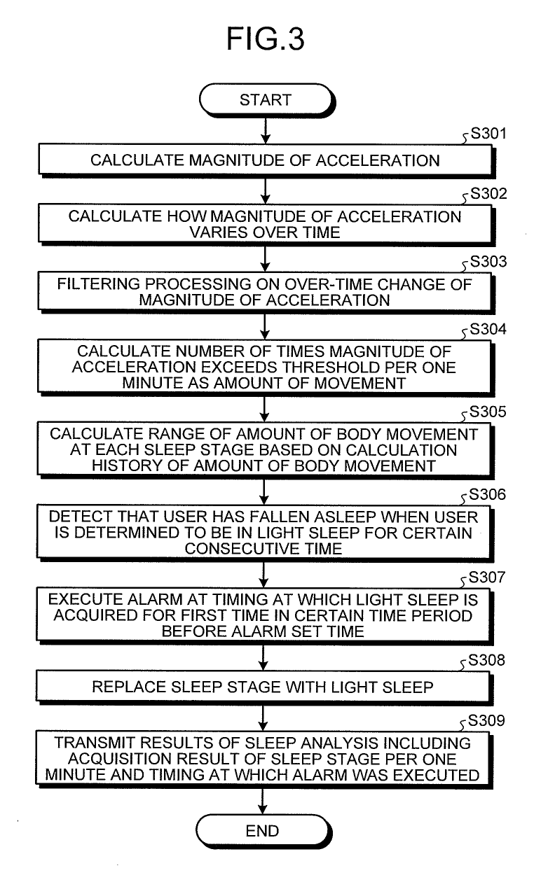

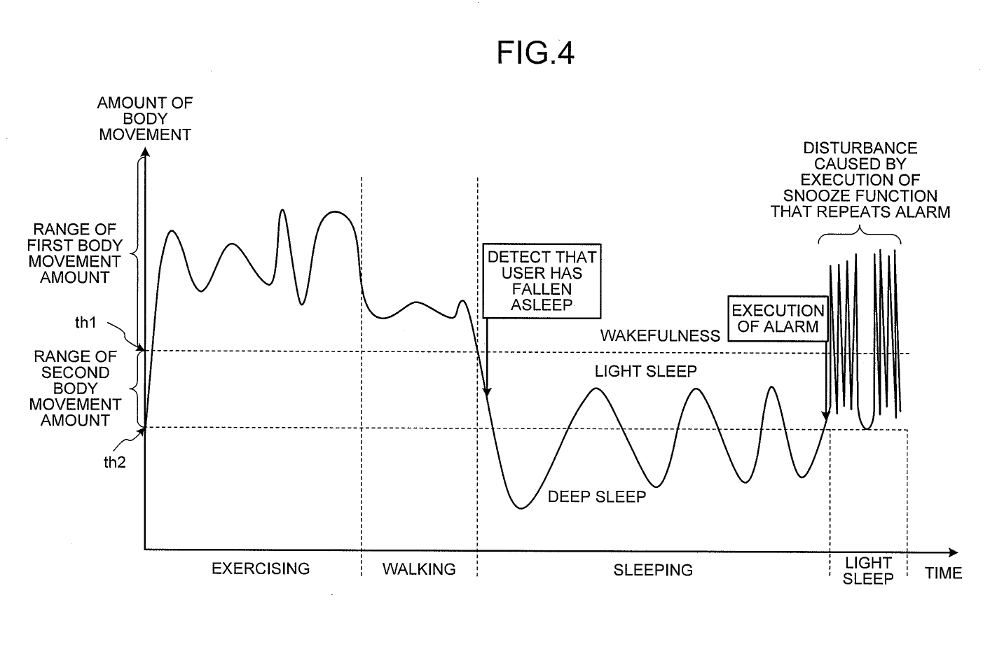

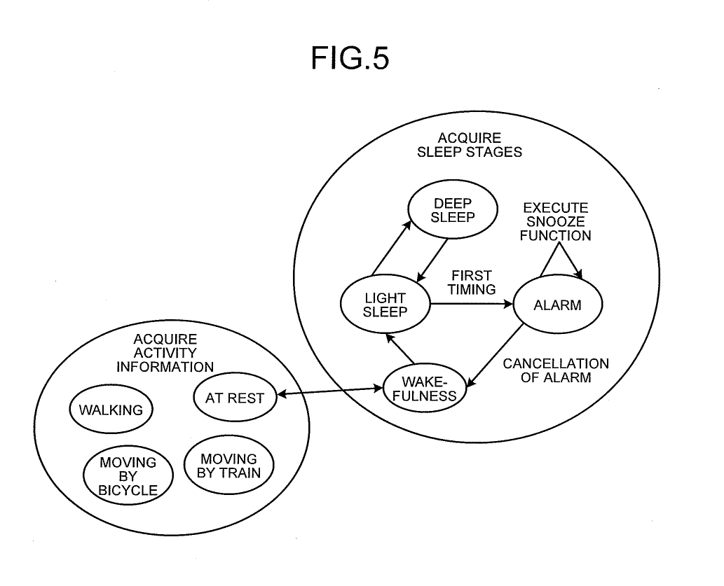

Next, described in detail is the acquisition processing for acquiring the body information by the wearable device 1 according to the present embodiment with reference to FIGS. 3 to 5. FIG. 3 is a flowchart illustrating an example of the procedure for acquiring the body information by the wearable device according to the present embodiment. FIG. 4 is a diagram illustrating an example of time variation of body movement amount calculated by the wearable device according to the present embodiment. FIG. 5 is a diagram illustrating an example of the acquisition processing for acquiring the body information by the wearable device according to the present embodiment.

When the wearable device 1 is powered on, the analysis controller 16 causes the accelerometer 12 to start detecting acceleration data. The body movement amount calculation module 15 calculates the magnitude of acceleration on the basis of the acceleration data detected by the accelerometer 12 (S301). In the present embodiment, when the accelerometer 12 is a three-axis accelerometer, the body movement amount calculation module 15 calculates magnitude of acceleration Acc_Norm[n] by use of an expression (1) below on the basis of x-axis acceleration data x[n], y-axis acceleration data y[n], and z-axis acceleration data z[n].

Acc_Norm[n]=\sqrt{x[n]²+y[n]²+z[n]²} (1)

The body movement amount calculation module 15 also calculates time variation of the calculated magnitude of acceleration (S302). In the present embodiment, the body movement amount calculation module 15 calculates diff_Acc_Norm[n] representing the time variation of the magnitude of acceleration by use of an expression (2) below.

diff_Acc_Norm[n]=Acc_Norm[n]−1.0 (2)

The body movement amount calculation module 15 performs filtering processing for removing noise (for example, time variation of the magnitude of acceleration of the housing 10 caused by external force other than body movement) contained in the calculated time variation of the magnitude of acceleration (S303). The body movement amount calculation module 15 calculates, on the basis of the calculated time variation of the magnitude of acceleration, the number of times the time variation of the magnitude of acceleration exceeds a certain threshold per unit time (for example, one minute) as an amount of body movement of the user (S304). The certain threshold is the lower limit of the magnitude of acceleration determined as a body movement of the user.

After the body movement amount calculation module 15 calculates an amount of body movement, the analysis controller 16 controls the activity analysis module 172 to start acquiring activity information that is an example of the body information. As illustrated in FIGS. 4 and 5, the activity analysis module 172 acquires the activity information (for example, the number of steps the user has taken, the user is exercising, walking, at rest, moving by bicycle, or moving by train) on the basis of the acceleration data detected by the accelerometer 12.

The analysis controller 16 calculates a range S[f] of the amount of body movement in each sleep stage used for determining the sleep stage on the basis of the calculation history of amounts of body movement calculated by the body movement amount calculation module 15 by use of a sleep estimation determination algorithm such as the Cole's algorithm represented by an expression (3) below or an AW2 algorithm represented by an expression (4) below (S305).

$$\begin{matrix}S [ f ] = 0.0001 \times \{ \begin{matrix}404 \cdot A [ f - 6 ] + 598 \cdot A [ f - 5 ] + 326 \cdot A [ f - 4 ] + \\ 441 \cdot A [ f - 3 ] + 1408 \cdot A [ f - 2 ] + 508 \cdot A [ f - 1 ] + \\ 350 \cdot A [ f ]\end{matrix} \} & ( 3 ) \\ S [ f ] = 0.0033 \times \{ \begin{matrix}1.06 \cdot A [ f - 6 ] + 0.54 \cdot A [ f - 5 ] + \\ 0.58 \cdot A [ f - 4 ] + 0.76 \cdot A [ f - 3 ] + 2.3 \cdot A [ f - 2 ] + \\ 0.74 \cdot A [ f - 1 ] + 0.67 \cdot A [ f ]\end{matrix} \} & ( 4 )\end{matrix}$$

In the present embodiment, the analysis controller 16 calculates a range of a first body movement amount by which the user is determined to be awake, a range of a second body movement amount by which the user is determined to be in a stage of light sleep and that is smaller than the first body movement amount, and a range of a third body movement amount by which the user is determined to be in a stage of deep sleep and that is smaller than the second body movement amount.

When the user operates the button 14 to input going to sleep, the sleep analysis module 171 detects that the user has fallen asleep. Alternatively, as illustrated in FIG. 4, when the sleep analysis module 171 determines that the amount of body movement calculated by the body movement amount calculation module 15 is consecutively smaller than a lower limit th1 of the range of the first body movement amount for a certain time period (for example, 20 minutes), that is, when the sleep analysis module 171 determines that the user is in light sleep (see FIG. 4), the sleep analysis module 171 detects that the user has fallen asleep (S306).

When the user operates the button 14 to input a first setting (in the present embodiment, bed time at which the user presses and holds the button 14 when the sleep analysis module 171 is not acquiring the sleep stages) indicating acquisition of the sleep stages that are an example of the body information, the sleep analysis module 171 detects that the user has fallen asleep on the basis of the timing at which the bed time is input. As illustrated in FIGS. 4 and 5, the sleep analysis module 171 then continues to acquire the sleep stage of the user per unit time on the basis of the amount of body movement calculated per unit time (for example, one minute) by the body movement amount calculation module 15. In the present embodiment, the sleep analysis module 171 acquires wakefulness as the sleep stage of the user when the amount of body movement calculated by the body movement amount calculation module 15 falls within the range of the first body movement amount (see FIGS. 4 and 5). The sleep analysis module 171 acquires light sleep as the sleep stage of the user when the amount of body movement calculated by the body movement amount calculation module 15 falls within the range of the second body movement amount (see FIGS. 4 and 5). The sleep analysis module 171 acquires deep sleep as the sleep stage of the user when the amount of body movement calculated by the body movement amount calculation module 15 falls within the range of the third body movement amount (in other words, when the sleep analysis module 171 determines that the amount of body movement calculated by the body movement amount calculation module 15 is smaller than a lower limit th2 of the range of the second body movement amount) (see FIGS. 4 and 5).

In the present embodiment, when the user does not input the bed time, the sleep analysis module 171 does not acquire the sleep stages of the user even when the sleep analysis module 171 detects that the user has fallen asleep.

As illustrated in FIG. 4, the alarm controller 18 causes the vibrator 13 and the speaker 700 to execute the alarm at the first timing (S307). As described above, the first timing is the earlier timing of the two timings, the alarm set time set by the alarm setting module 19 and a timing at which the sleep analysis module 171 acquires light sleep as the sleep stage of the user for the first time in a certain time period prior to the alarm set time. The alarm controller 18 causes the vibrator 13 to execute the alarm in accordance with a certain vibration pattern. The alarm controller 18 may set in advance, as the certain vibration pattern, a vibration pattern candidate selected from a plurality of vibration pattern candidates through the button 14 or the host device 2.

While the vibrator 13 and the speaker 700 are executing the alarm, the alarm controller 18 displays, on the display screen 11, the display information relating to the reason why the alarm is being executed. Specifically, the alarm controller 18 displays the display information indicating, for example, that the first timing has come, or that the notification information such as incoming call or emergency call is received from the host device 2.

When the alarm is being executed because the first timing has come, the alarm controller 18 can control the vibrator 13 and the speaker 700 to execute a snooze function that repeats the alarm certain times or for a certain time period set in advance as illustrated in FIG. 4. The alarm controller 18 controls the vibrator 13 and the speaker 700 to continue the snooze function until the user operates the button 14 to cancel the snooze function. By this configuration, the snooze function never stops until the user operates the button 14 to input cancellation of the snooze function, thereby preventing the user who wears the wearable device 1 from sleeping in again.

When the vibrator 13 and the speaker 700 are executing the alarm, the acceleration data detected by the accelerometer 12 contains disturbance caused by the alarm executed by the vibrator 13 and the speaker 700. Thus, as illustrated in FIG. 4, the amount of body movement calculated by the body movement amount calculation module 15 also contains the disturbance caused by the alarm executed by the vibrator 13 and the speaker 700. Consequently, the sleep analysis module 171 cannot acquire the sleep stages of the user accurately because of the abnormal acceleration data detected by the accelerometer 12, which causes, for example, the sleep analysis module 171 to incorrectly determine that the user is awake. When the alarm controller 18 has a function to automatically stop the alarm on acquiring that the user is awake by the sleep analysis module 171, the alarm is stopped even though the user is still sleeping.

In the present embodiment, the analysis controller 16 replaces the sleep stage acquired by the sleep analysis module 171 while the vibrator 13 and the speaker 700 are executing the alarm with a certain sleep stage (for example, light sleep) (S308). When an amount of body movement is acquired as an example of the body information, the analysis controller 16 replaces the amount of body movement calculated by the body movement amount calculation module 15 while the vibrator 13 and the speaker 700 are executing the alarm with a certain amount of body movement that is an example of a certain second information different from the calculated amount of body movement. The certain amount of body movement described above is an amount of body movement calculated by the body movement amount calculation module 15 a first certain amount of time before the execution of the alarm by the vibrator 13 and the speaker 700.

When acceleration data is acquired as an example of the body information, the analysis controller 16 replaces the acceleration data detected by the accelerometer 12 while the vibrator 13 and the speaker 700 are executing the alarm with certain acceleration data that is an example of the certain second information different from the detected acceleration data. The certain acceleration data described above is acceleration data detected by the accelerometer 12 the first certain amount of time before the execution of the alarm by the vibrator 13 and the speaker 700.

After stopping (canceling) the alarm executed by the vibrator 13 and the speaker 700 depending on indication input from the button 14, when the acquired sleep stage is not wakefulness yet, the analysis controller 16 makes the sleep analysis module 171 continue to acquire the sleep stages until the sleep analysis module 171 acquires wakefulness. The acquisition module 20 acquires information relating to the time from the execution of the alarm by the vibrator 13 and the speaker 700 (that is, the first timing) to the time at which the sleep analysis module 171 acquires wakefulness as the sleep stage of the user as time information required for the user to wake up.

The communication module 21 transmits body information including the acceleration data detected by the accelerometer 12, the amount of body movement calculated by the body movement amount calculation module 15, and results of the sleep analysis to the host device 2 (S309). The results of the sleep analysis include the bed time, the sleep stage per unit time acquired by the sleep analysis module 171, the timing at which the alarm was executed (that is, the first timing), the sleep time at which the sleep analysis module 171 detected that the user had fallen asleep after the bed time was input, the wake time at which the user pressed and held the button 14 after the sleep analysis module 171 started acquiring the sleep stages, the sleep time that is the sum of an amount of time during which light sleep was acquired and an amount of time during which deep sleep was acquired, light-sleep time in which the sleep analysis module 171 acquired light sleep, deep-sleep time in which the sleep analysis module 171 acquired deep sleep, and the time information required for the user to wake up.

The host device 2 transmits the results of the sleep analysis received from the communication module 21 to the server 3. The server 3 analyzes the results of the sleep analysis received from the host device 2 and transmits results of the analysis back to the host device 2. The host device 2 displays the results of the analysis received from the server 3 on the display screen 200.

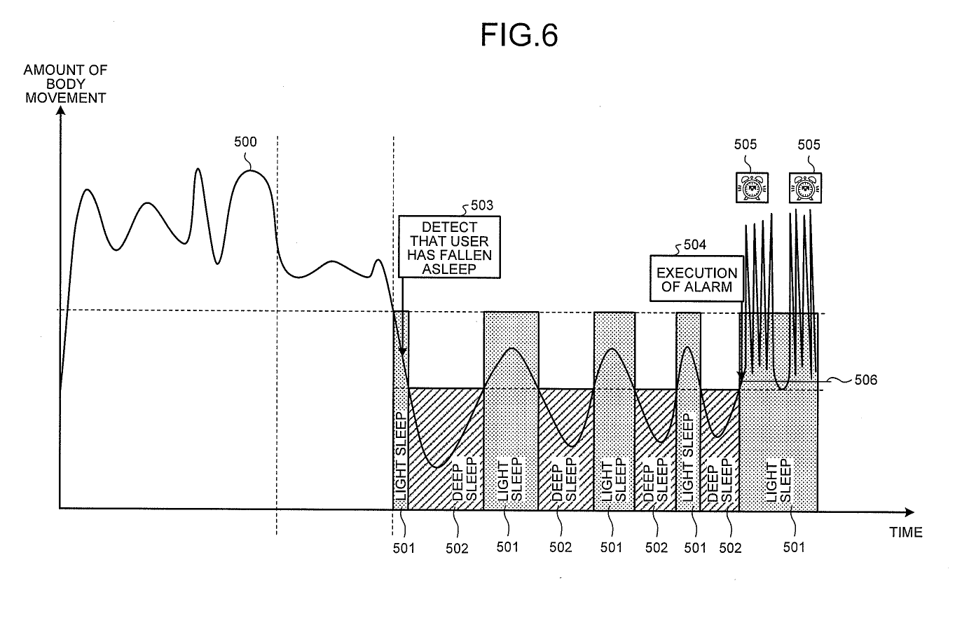

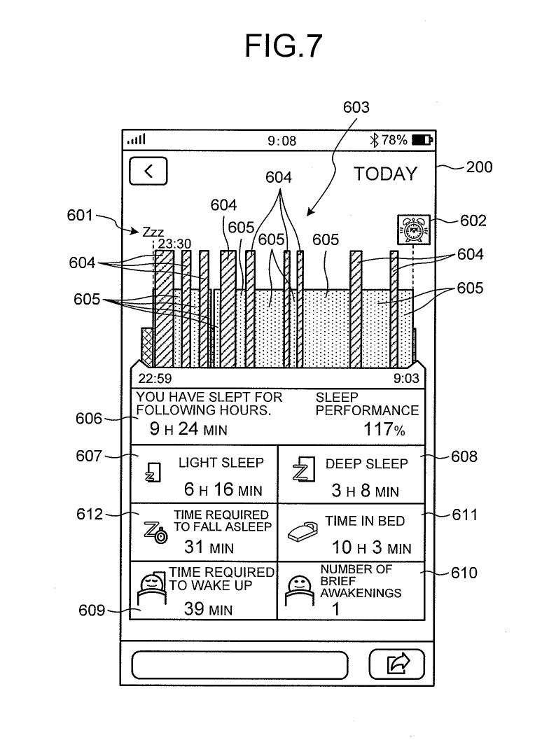

Described next is a display example of the results of the analysis displayed on the host device 2 with reference to FIGS. 6 and 7. FIG. 6 is a diagram illustrating a display example of the results of the analysis displayed on the host device according to the present embodiment. FIG. 7 is a diagram illustrating another display example of the results of the analysis displayed on the host device according to the present embodiment.

In the present embodiment, as illustrated in FIG. 6, the host device 2 displays, on the display screen 200, the results of the analysis including a graph 500 indicating the time variation of the amount of body movement calculated by the body movement amount calculation module 15 per unit time, a light-sleep indicator 501 indicating a time period in which the sleep analysis module 171 acquired light sleep, a deep-sleep indicator 502 indicating a time period in which the sleep analysis module 171 acquired deep sleep, a sleep time indicator 503 indicating the sleep time at which the sleep analysis module 171 detected that the user had fallen asleep after the bed time was input, an alarm start indicator 504 indicating the first timing at which the alarm started, an alarm execution indicator 505 indicating a time period during which the alarm was executed, and a replaced time indicator 506 indicating a period in which a sleep stage acquired by the sleep analysis module 171 was replaced with light sleep.

Alternatively, in the present embodiment, as illustrated in FIG. 7, the host device 2 displays, on the display screen 200, the results of the analysis including a sleep time indicator 601 indicating the sleep time at which the sleep analysis module 171 detected that the user had fallen asleep after the bed time was input, an alarm start indicator 602 indicating the first timing at which the alarm started, and a sleep stage indicator 603 including a light-sleep indicator 604 indicating a time period in which the sleep analysis module 171 acquired light sleep and a deep-sleep indicator 605 indicating a time period in which the sleep analysis module 171 acquired deep sleep that are arranged in time order.

As illustrated in FIG. 7, the host device 2 may display, on the display screen 200, the results of the analysis including total sleep time 606 that is the sum of an amount of time in, which the sleep analysis module 171 acquired light sleep and an amount of time in which the sleep analysis module 171 acquired deep sleep, light-sleep time 607 that is an amount of time during which the sleep analysis module 171 acquired light sleep, deep-sleep time 608 that is an amount of time during which the sleep analysis module 171 acquired deep sleep, time 609 required for the user to wake up, a number of brief awakenings 610 that is the number of times the user briefly woke up during the time period from the sleep time to the wake time, time in bed 611 that is an amount of time from the sleep time at which the sleep analysis module 171 detected that the user had fallen asleep to the wake time, and time 612 required for the user to fall asleep from the bed time to the sleep time.

The wearable device 1 according to the present embodiment can prevent disturbance caused by the alarm executed by the vibrator 13 and the speaker 700 from being included in the sleep stages acquired by the sleep analysis module 171, thereby improving accuracy of the sleep stages acquired by the wearable device 1.

Although, in the above description, the wearable device 1 according to the present embodiment replaces the body information (acceleration data, an amount of body movement, sleep stages, activity information) acquired by the accelerometer 12, the body movement amount calculation module 15, and the analysis module 17 with a certain second information, the wearable device 1 can replace other types of body information with a certain second information.

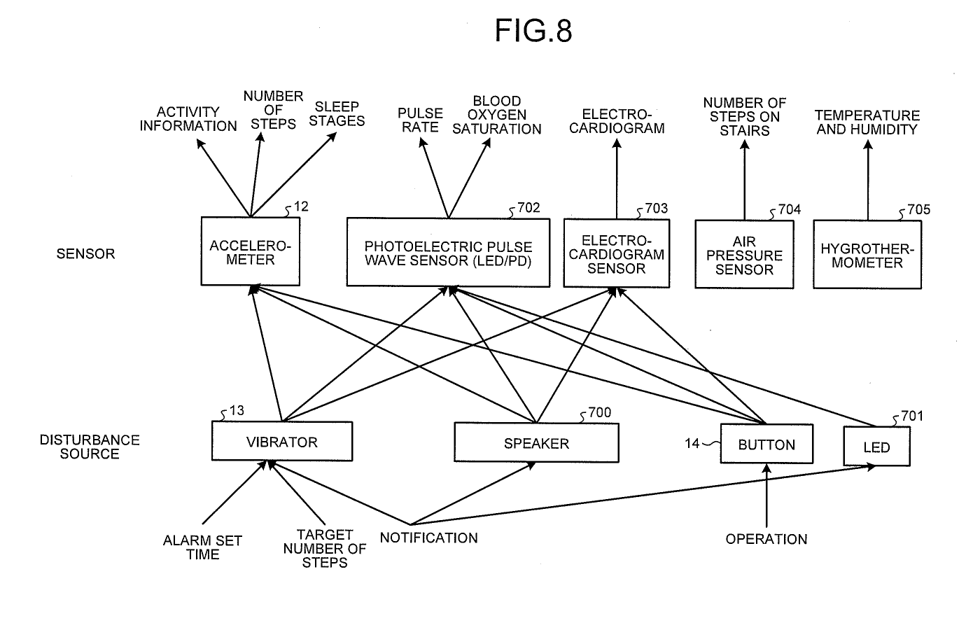

FIG. 8 is a diagram illustrating an example of replacement of other types of the body information with certain second information by the wearable device according to the present embodiment. As illustrated in FIG. 8, the analysis controller 16 may replace body information detected by various types of sensors (for example, the accelerometer 12, a photoelectric pulse wave sensor 702, an electrocardiogram sensor 703, an air pressure sensor 704, and a hygrothermometer 705) with certain second information depending on whether the body information detected by these sensors is of a type of body information that may be subject to the disturbance caused by the first operation of a disturbance source (for example, the vibrator 13, the speaker 700, the button 14, and a light emitting diode (LED) 701).

As illustrated in FIG. 8, for example, when the wearable device 1 acquires body information that may be subject to disturbance caused by the first operation of the disturbance source, such as the activity information, the number of steps, and the sleep stages based on acceleration data detected by the accelerometer 12, a pulse rate and blood oxygen saturation that are an example of the body information detected by the photoelectric pulse wave sensor 702 including the LED and a photo diode (PD), and an electrocardiogram that is an example of the body information detected by the electrocardiogram sensor 703, the analysis controller 16 replaces the body information such as the activity information, the number of steps, the sleep stages, the pulse rate, the blood oxygen saturation, and the electrocardiogram acquired while the first operation is being executed with certain second information (certain activity information, a certain number of steps, a certain sleep stage, a certain pulse rate, a certain blood oxygen saturation, a certain electrocardiogram) that is different from the acquired body information.

Meanwhile, as illustrated in FIG. 8, when the wearable device 1 acquires body information that is not subject to disturbance caused by the first operation of the disturbance source, such as the number of steps the user takes when going upward or downward on stairs based on air pressure detected by the air pressure sensor 704, and the temperature and humidity detected by the hygrothermometer 705, the analysis controller 16 prohibits the replacement of the body information such as the number of steps on stairs and the temperature and humidity acquired while the first operation is being executed with certain second information.

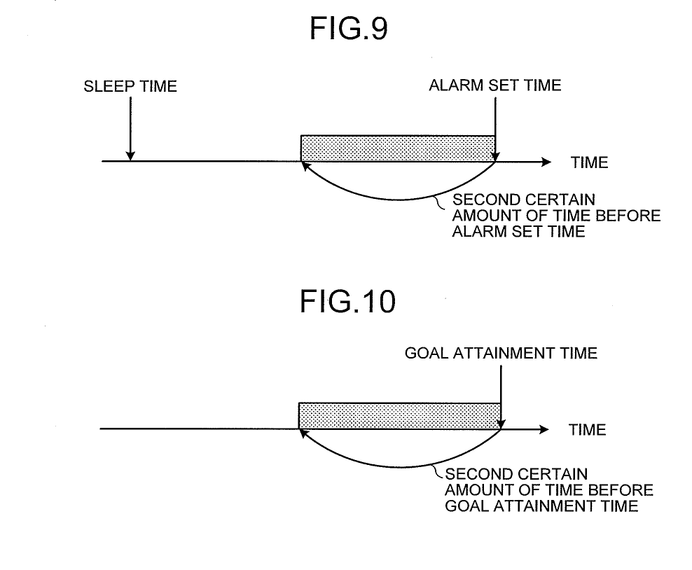

Although, in the present embodiment, when the first setting (in the present embodiment, the bed time) indicating the acquisition of the sleep stages that are an example of the body information is not input through the button 14, the analysis controller 16 causes the sleep analysis module 171 not to acquire the sleep stages of the user, the embodiment is not limited to this. FIGS. 9 and 10 are diagrams illustrating an example of acquisition processing for acquiring the body information by the wearable device according to the present embodiment. As illustrated in FIG. 9, for example, when the bed time was not input to the wearable device 1, the analysis controller 16 may cause the sleep analysis module 171 to start acquiring the sleep stages a second certain amount of time before the first timing (for example, the alarm set time). With this configuration, even when the user forgets inputting the bed time on the wearable device 1 before falling asleep, the wearable device 1 can acquire the sleep stages. Thus, the user can acquire the results of sleep analysis when the user forgets inputting the bed time.

When the number of steps based on the acceleration data detected by the accelerometer 12 is acquired as an example of the body information, the analysis controller 16 causes the activity analysis module 172 to start acquiring the number of steps on the basis of a timing at which the user inputs the first setting indicating the acquisition of the number of steps that is an example of the body information through the button 14. As illustrated in FIG. 10, when the user does not input the first setting indicating the acquisition of the number of steps through the button 14, the analysis controller 16 may cause the activity analysis module 172 to start acquiring the number of steps the second certain amount of time before a goal attainment time at which the user is estimated to achieve the target number of steps. With this configuration, even when the user of the wearable device 1 forgets inputting the first setting before the user started walking, the wearable device 1 acquires the number of steps. Thus, the user can acquire the number of steps when the user forgets inputting the first setting.

Although, in the present embodiment, the analysis controller 16 replaces the acceleration data detected by the accelerometer 12 while the alarm is being executed by the vibrator 13 with certain acceleration data different from the detected acceleration data, the embodiment is not limited to this. The analysis controller 16 may perform, for example, processing (what is called noise cancelling processing) for eliminating, from the acceleration data detected by the accelerometer 12 while the alarm is being executed by the vibrator 13, the disturbance caused by the alarm.

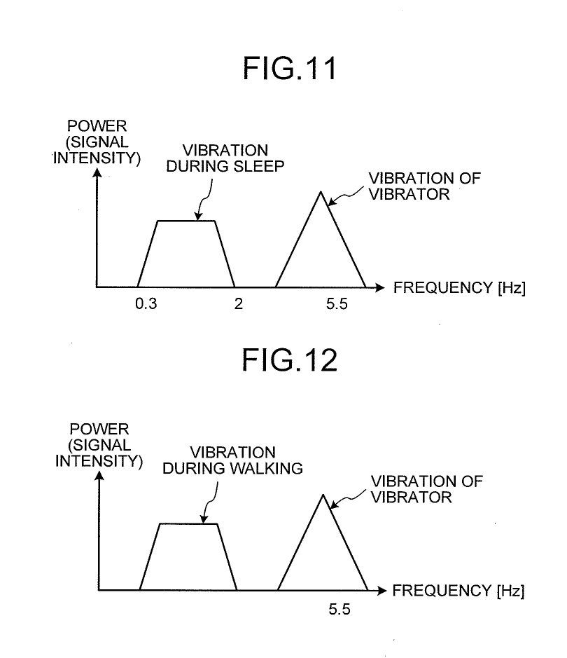

FIG. 11 is a diagram illustrating an example of the relation between the intensity of a signal of acceleration data and the frequency of the signal of the acceleration data that varies over time when the wearable device according to the present embodiment acquires the sleep stages of a user. FIG. 12 is a diagram illustrating an example of the relation between the intensity of a signal of acceleration data and the frequency of the signal of the acceleration data when the wearable device according to the present embodiment acquires the number of steps of the user. In FIGS. 11 and 12, the vertical axis represents the intensity (power) of a signal of acceleration data, and the horizontal axis represents the frequency of the signal of the acceleration data. As illustrated in FIG. 11, the frequency of a signal of acceleration data that varies over time by vibration of the user while sleeping differs from the frequency of a signal of acceleration data that varies over time by vibration of the vibrator 13. As illustrated in FIG. 12, the frequency of a signal of acceleration data that varies over time by vibration of the user while walking differs from the frequency of a signal of acceleration data that varies over time by vibration of the vibrator 13. The analysis controller 16 performs noise cancelling processing that removes a frequency corresponding to the frequency of vibration of the vibrator 13 from a signal of the acceleration data detected by the accelerometer 12. With this processing, the wearable device 1 can eliminate disturbance caused by the vibrator 13 from acceleration data detected by the accelerometer 12 and body information acquired on the basis of the acceleration data.

The analysis controller 16 does not necessarily perform the noise cancelling processing on all the acceleration data detected by the accelerometer 12 while the user is sleeping or walking. The analysis controller 16 may start the noise cancelling processing on the acceleration data detected by the accelerometer 12 the second certain amount of time before the alarm set time illustrated in FIG. 9 (or before the goal attainment time illustrated in FIG. 10).

Moreover, the various modules of the systems described herein can be implemented as software applications, hardware and/or software modules, or components on one or more computers, such as servers. While the various modules are illustrated separately, they may share some or all of the same underlying logic or code.

While a certain embodiment has been described, the embodiment has been presented by way of example only, and is not intended to limit the scope of the invention. Indeed, the novel embodiment described herein may be embodied in a variety of other forms; furthermore, various omissions, substitutions, and changes in the form of the embodiment described herein may be made without departing from the spirit of the invention. The accompanying claims and their equivalents are intended to cover such forms or modifications as would fall within the scope and spirit of the invention.

What is claimed is:

1. An electronic device comprising:
an indicator configured to notify a user by using at least one of a sound and vibration;
a sensor configured to detect first information regarding a body of the user, the first information possibly subject to disturbance during notifying by the indicator; and
circuitry configured to replace the first information during notifying by the indicator with second information different from the first information.

2. The electronic device of claim 1, wherein the second information is equal to the first information detected by the sensor a first certain amount of time before notifying by the indicator.

3. The electronic device of claim 1, wherein the circuitry is further configured to prohibit replacement of the first information with the second information when a type of the first information detected by the sensor corresponds to a type of information that is not subject to disturbance caused by notifying by the indicator.

4. The electronic device of claim 1, wherein
the indicator is further configure to notify the user by using at least one of the sound and vibration at a certain first timing, and
when a first setting indicative of detection of the first information is input, the circuitry is further configured to cause the sensor to start detecting the first information after the first setting is input, and, when the first setting is not input, the circuitry is further configured to cause the sensor to start detection the first information at a timing a second certain amount of time before the first timing.

5. The electronic device of claim 1, wherein
the first information is information relating to a sleep stage of the user,
the indicator is further configured to execute rumbling,
the sensor is further configured to continue, after the rumbling is stopped, to detect the first information until the sensor senses wakefulness as the sleep stage of the user, and
the circuitry is further configured to output information relating to an amount of time from execution of the rumbling to sensing of wakefulness as the sleep stage based on the first information by the sensor, as time information required for the user to wake up.

6. A method performed by an electronic device comprising:
notifying a user by using at least one of a sound and vibration;
detecting first information that regarding a body of the user, the first information possibly subject to disturbance during the notification; and
replacing the first information during the notification with second information different from the first information.

7. The method of claim 6, wherein the second information is equal to the first information detected a first certain amount of time before the notification.

8. The method of claim 6, further comprising:
prohibiting replacement of the first information with the second information when a type of the detected first information corresponds to a type of information that is not subject to disturbance caused by the notification.

9. The method of claim 6, wherein
the notification is executed at a certain first timing, and
when a first setting indicative of detection of the first information is input, the detection of the first information is started after the first setting is input, and when the first setting is not input, the detection of the first information is started at a timing a second certain amount of time before the first timing.

10. The method of claim 6, wherein
the first information relates to a sleep stage of the user,
rumbling is executed as the notification, and
after the rumbling is stopped, the detection of the first information is continued until wakefulness is sensed as the sleep stage of the user,
the method further comprising:
outputting information relating to an amount of time from execution of the rumbling to sensing of wakefulness as the sleep stage based on the detected first information, as time information required for the user to wake up.

11. A computer program product having a non-transitory computer readable medium including programmed instructions, wherein the instructions, when executed by a computer, cause the computer to perform:
notifying a user by using at least one of a sound and vibration;
detecting first information that regarding a body of the user, the first information possibly subject to disturbance during notification; and
replacing the first information during the notification with second information different from the first information.

12. The computer program product of claim 11, wherein the second information is equal to the first information detected a first certain amount of time before the notification.

13. The computer program product of claim 11, wherein the replacing of the first information with the second information is prohibited when a type of the detected first information corresponds to a type of information that is not subject to disturbance caused by the notification.

14. The computer program product of claim 11, wherein
the notification is executed at a certain first timing,
when a first setting indicative of the detection of the first information is input, the detection of the first information is started after the first setting is input, and when the first setting is not input, the detection of the first information is started at a timing a second certain amount of time before the first timing.

15. The computer program product of claim 11, wherein
the first information relates to a sleep stage of the user,
rumbling is executed as the notification,
after the rumbling is stopped, the detection of the first information is continued until wakefulness is sensed as the sleep stage of the user, and
outputting information relating to an amount of time from execution of the rumbling to sensing of wakefulness as the sleep stage based on the detected first information, as time information required for the user to wake up.
